(19) United States
(12) Patent Application Publication
(10) Pub. No.: US 2011/0171688 A1
(43) Pub. Date: Jul. 14, 2011

(54) METHOD OF PROVIDING IMMUNOGLOBULIN SECRETING B LYMPHOCYTES AND HUMAN ANTIBODIES

(76) Inventor: Christoph Esslinger, Zurich (CH)

(21) Appl. No.: 12/991,422

(22) PCT Filed: Jun. 16, 2009
(86) PCT No.: PCT/EP09/04365

§ 371 Date: Feb. 8, 2011

(30) Foreign Application Priority Data
Jun. 16, 2008 (EP) 08010929.1

(51) Int. Cl. C12P 21/08; C12N 5/10; C07K 16/00

(52) U.S. Cl. 435/69.6; 435/325; 530/388.1; 530/388.15

(57) ABSTRACT

Provided is a method for producing a clone of an immortalized human B memory lymphocyte, comprising the step of inducing or enhancing telomerase activity in the B lymphocyte in the presence of a polyclonal B cell activator. The method is particularly useful in a method for producing a clone of an immortalized human B memory lymphocyte capable of producing a human monoclonal antibody with desired antigen specificity.

### FIELD OF THE INVENTION

The present invention relates to a method for inducing proliferation, secretion of immunoglobulin and prolongation of the life span of human memory B cells in vitro, comprising a transformation step of memory B cells by a system which provides human Telomerase-Reverse-Transcriptase activity preferably combined with a polyclonal B cell activator. The present invention is particularly useful for preparing human monoclonal antibodies.

### BACKGROUND OF THE INVENTION

Human monoclonal antibodies are of pharmaceutical interest as candidates for immunotherapy in a variety of therapeutic indications. Current approaches comprise the immunization of transgenic mice that contain parts of the human immune system, or selection in vitro using human immune or non-immune libraries (phage display). A direct approach to obtain a potentially valuable antibody would be by direct cloning of selected B cells from a human donor that expresses an antibody with a desired specificity. Antibodies from such a B cell clone would be most interesting if memory B cells would be used as the source because these cells bear the potential to produce affinity matured antibodies.

The typical methods established suffer from the drawback that they are not suitable to produce antibodies with the characteristics of those produced in the course of a physiological human immune response. Antibodies that occur naturally in humans either in response to an intrinsic pathogenic stimulus or exposure to an infectious agent may be directed against previously unidentified epitopes. Moreover, targets identified by such antibodies may be of higher therapeutic relevance than those antibodies that had been generated to targets that were selected based on scientifically biased assumptions about a potential therapeutic importance. In addition, the origin and maturation in a human subject of such human derived antibodies can be supposed to significantly decrease the probability of undesirable off-target reactivity and auto-toxicity in humans because the clinical history of the subject of origin can be selected to ensure the absence of undesirable side effects.

Techniques for the cloning of antibodies directly from a human subject have been described, these include the hybridoma technique or the EBV immortalization technique, or a combination of both (Steinitz et al., Nature 269 (1977), 420-422; Kozbor et al., J. Immunol. 127 (1981), 1275-1280 and Roder et al., Methods Enzymol. 121 (1986), 140-167). All these techniques suffer from the disadvantage of being of low efficiency. A more recent improvement of the EBV immortalization method has been described by Traggiai et al., Nature Medicine 10 (2004), 871-875. This method suffers from clonal instability most probably due to continued somatic hypermutation of antibody genes.

Therefore, an immunoglobulin providing system would be desirable that leads to the efficient transformation of resting memory B cells into immunoglobulin-secreting cells (plasma cells) which are stable over a prolonged life span and produce antibodies that maintain the desired target-specificity for a certain period of time. This period comprises the time necessary to screen for the antibody specificity and the subsequent cloning of those cells with a specificity of interest.

The solution to this technical problem is provided by the embodiments characterized in the claims and described further below.

### SUMMARY OF THE INVENTION

The present invention generally relates to a method of preparing monoclonal antibodies and equivalent antigen-binding molecules comprising producing a B lymphocyte of prolonged life span by inducing or enhancing telomerase activity in the B lymphocyte, in particular in a human memory B cell. While the present invention is illustrated by embodiments where human monoclonal antibodies are produced, the techniques described herein are not so limited. The present invention can be used for any species for which it is desired to produce monoclonal antibodies efficiently.

The present invention is based on the observation that heterologous expression of human Telomerase-Reverse-Transcriptase (hTert) induces prolongation of life span, secretion of immunoglobulin and clonal growth in human memory B lymphocytes in vitro enabling the characterization of antibody specificity on a clonal level. Selected cells can then be used for monoclonal antibody production. This method preferably does not involve cellular fusion of the B memory lymphocytes with other cells.

Thus, in one embodiment telomerase activity in the B lymphocyte is induced or enhanced by introducing into the B lymphocyte a nucleic acid molecule encoding a polypeptide having telomerase activity, for example Telomerase-Reverse-Transcriptase (Tert), or a catalytically active fragment or derivative thereof. Typically, the nucleic acid molecule is contained in a vector, preferably a lentiviral vector.

In a preferred embodiment the method of the present invention comprises culturing the B lymphocyte in the presence of a polyclonal B cell activator. For example, the nucleic acid molecule encoding the polypeptide having telomerase activity is transfected in combination with a polyclonal B cell activator. The polyclonal B cell activator can be a CpG oligodeoxynucleotide among others. However, preferably CpG 2006 is used.

In a further embodiment the method of the present invention comprises culturing the B lymphocyte in the presence of a stimulant of cellular growth and/or differentiation, for example a cytokine, preferably IL-2 or IL-15.

In a preferred embodiment of the method of the present invention a subpopulation of B lymphocytes having antigen specificity is selected before inducing or enhancing telomerase activity. In principle, any desired antigen may be selected including but not limited to the group consisting of a human pathogen, toxin, chemical compound, allergen, tumor antigen, autoantigen, alloantigen or neoepitope of an otherwise physiological protein. Most preferably, the antigen is involved in Alzheimer's disease or cancer.

In a particular preferred embodiment of the method of the present invention the B lymphocyte is derived from a sample obtained from a subject who is symptom-free but affected with or at risk of developing a disorder, or a patient with an unusually stable disease course.

As described in the appended examples, the method of the present invention in order to produce a clone of a substantially immortalized human memory B cell capable of producing a human monoclonal antibody with a desired antigen specificity is preferably performed by:
(i) transforming a population of cells comprising or consisting of human memory B lymphocytes with a Lentivirus virus encoding a polypeptide providing Telomerase-Reverse-Transcriptase activity in the presence of a polyclonal B cell activator;
(ii) screening the culture supernatant for antigen specificity; and
(iii) isolating a human memory B cell clone of prolonged life time for several or multiple cycles of replication capable of producing a human monoclonal antibody having the desired antigen specificity.

The cloning is preferably carried out using limiting dilution.

In a still further embodiment the method of the present invention comprises the steps of:
(i) purifying the B lymphocytes from a sample which has been identified to express an antibody of desired specificity;
(ii) obtaining the immunoglobulin gene repertoire for said antibody from the B lymphocytes; and
(iii) using said repertoire to express the antibody; and optionally
(iv) obtaining mRNA from the B lymphocytes;
(v) obtaining cDNA from the mRNA of step (iv); and
(vi) using a primer extension reaction to amplify from said cDNA the DNA fragments corresponding to the immunoglobulin heavy chains (HC) and the kappa light chains (LC) of the antibody.

Subsequently, the DNA fragments may be cloned into an expression host cell in order to permit expression of the antibody of interest or immunoglobulin chain thereof in that host cell. Naturally, the nucleic acid molecule can be manipulated between steps (ii) and (iii) to introduce restriction sites, to change codon usage, alter the amino acid sequence of the immunoglobulin chain while keeping antigen specific in kind and/or to add or optimize transcription and/or translation regulatory sequences.

In a further embodiment the method of the present invention comprises culturing the host cell under conditions where the antibody of interest is expressed; and optionally purifying the antibody the interest or immunoglobulin chain thereof.

In a particular preferred embodiment the method of the present invention comprises the steps of:
(a) transforming a human memory B cell into a substantially immortalized immunoglobulin secreting cell, comprising
   (i) introducing human Telomerase-Reverse-Transcriptase (hTert) through lentivector-mediated gene transfer into human blood-derived memory B cells;
   (ii) in the presence of a polyclonal B cell activator;
(b) selecting a B cell of prolonged life span that produces an antibody with a desired specificity;
(c) obtaining and/or sequencing a nucleic acid molecule from the selected B cell encoding at least the binding domain of the antibody of interest;
(d) inserting the nucleic acid molecule into or using the nucleic acid sequence to prepare an expression host cell that is capable of expressing the antibody of interest or an immunoglobulin chain thereof;
(e) culturing or sub-culturing the expression host cell under conditions where the antibody of interest is expressed; and, optionally,
(f) purifying the antibody of the interest or immunoglobulin chain thereof.

In a further aspect the present invention relates to a substantially immortalized B lymphocyte clone obtainable by the method of the present invention described herein. The B lymphocyte clone of the present invention is preferably characterized by telomerase activity or an increased expression or activity of telomerase compared to a B lymphocyte which has not been subjected to the method of the present invention. In addition, or alternatively, the B lymphocyte clone of the present invention is characterized by the presence of a foreign nucleic acid molecule encoding a polypeptide having telomerase activity, for example Telomerase-Reverse-Transcriptase (Tert), or a catalytically active fragment or derivative thereof.

Naturally, the present invention also extends to the antibody or equivalent antigen-binding molecule obtainable by the method of the present invention, which antibody is preferably a human antibody. Included in the scope of the present invention are compositions of matter and kit-of-parts comprising a nucleic acid encoding a polypeptide having telomerase activity, for example Telomerase-Reverse-Transcriptase (Tert), or a catalytically active fragment or derivative thereof and a polyclonal activator, for example a CpG oligodeoxynucleotide such as CpG 2006, and optionally a B lymphocyte or reagents for the selection of a B lymphocyte. In addition, the composition and kit-of-parts of the present invention, respectively, may further comprise an antigen and/or a cytokine as mentioned above.

In a still further aspect the present invention relates to the use of a lentivector for the production of a substantially immortalized immunoglobulin secreting B lymphocyte.

Further embodiments of the present invention will be apparent from the description and Examples that follow. Furthermore, the description of the present invention, where necessary or appropriate, may be supplemented with the disclosure content of applicant's earlier international applications WO2008/081008 and WO2008/110373.

### BRIEF DESCRIPTION OF THE DRAWINGS

FIG. 1: IgG secretion by cultured human memory B cells transduced by hTert-expressing lentivector. Human IgG content in 8 individual 96 well cultures of hTert-transduced memory B cells (hTert) is shown in comparison to cultures of EBV transformed (EBV) and untreated (control) memory B cell cultures. B cell conditioned medium was used as a 10-fold dilution in PBS. As standard, purified human IgG was used at concentrations of 5 nM, 1.25 nM, 0.3 nM and 0.08 nM.

FIG. 2: Detection of B cell cultures secreting antibodies to Tetanus Toxoid (TT) and cellular cloning of hTert-transduced memory B cells producing antibodies reactive with TT. A) Screening of two 96 well plates containing cultures of hTert-lentivector transduced B cells was assayed in TT-ELISA. Non-specific binding was assayed in ELISA using BSA-coated plates (Mock). Several cultures display a specific reactivity to TT (arrows, bold black arrows show cultures selected for cloning). B) Cells from selected memory B cell cultures 6B7 and 7B4 were cloned by single-cell deposition using a cell sorter into 96 well plates. Medium conditioned by the cultures was analyzed 4 weeks later for the presence of antibodies specific for tetanus toxoid by ELISA.

FIG. 3: Increased telomerase activity in memory B cells transduced with hTert-expressing lentivector. Telomerase activity was measured in human B cell lines established upon transduction with hTert-expressing lentivector using a PCR-ELISA Telomeric Repeat Amplification Protocol (TRAP). Telomerase activity is shown in hTert-transduced memory B cell clones 6B7 and 7B4, untreated memory B cells (mBC) and in an EBV-transformed memory B cell line (EBV). As controls, the telomerase activity in heat inactivated cells (negative control) and in the carcinoma cell line HEK 293T (positive control) is shown.

### DETAILED DESCRIPTION OF THE INVENTION

Object of the present invention is a method for the cloning of human memory B cells that express antibodies that are specific for an antigen of interest, e.g. an antigen of pathological implication. A key step of the invention is the immortalization or the prolongation of the life span of human memory B cells in culture and their transformation into immunoglobulin-secreting cells mediated by ectopic expression of human Telomerase-Reverse-Transcriptase (hTert). This expression is achieved by lentivector-mediated gene transfer of hTert, the catalytic protein subunit of human telomerase into blood-derived memory B cells. In this context, it is noted that the method of the present invention does not need to provide complete immortalization of the cells, particularly, as their producer quality may not be expected to be suitable for commercial applications. Rather, it is sufficient to establish continuously replicating B cell lines for at least a period of time sufficient for determining antibody specificity and cloning by limiting dilution.

Hence, the present invention generally relates to a method of preparing monoclonal antibodies and equivalent antigen-binding molecules comprising producing a B lymphocyte of prolonged life span by inducing or enhancing telomerase activity in the B lymphocyte, in particular in a human memory B cell. Typically, the method comprises subsequent identification and cloning of B lymphocytes that produce the antibody of desired specificity.

Telomerase is a ribonucleoprotein responsible for the template independent synthesis of telomeric DNA. Ectopic expression of telomerase may prevent the physiologically normal cell senescence caused by telomere shortening during multiple cell division cycles (Meyerson et al., Cell 90 (1997), 785-95 and Nakamura et al., Science 277 (1997), 955-959).

Typically, the nucleic acid molecule encoding the polypeptide having telomerase activity, for example hTert is contained in a vector, preferably a lentiviral vector. Transduction of human memory B cells by lentivectors bearing a hTert expression cassette results in their prolonged survival, their clonal growth in culture and their secretion of immunoglobulin into the medium; see the appended Examples. This secretion facilitates the identification of such B cell clones that produce an antibody of interest. Monoclonal B cell lines can be established from hTert-transduced human memory B cells by cellular cloning. Human monoclonal antibodies (huMab) can then be obtained from the conditioned medium of clonal cell lines. Alternatively, such monoclonal antibodies could be obtained upon molecular cloning of such huMab encoding cDNAs or fragments of such cDNAs using well established methods. To that end, a small amount of cells, preferably clonally expanded cells, can be harvested, their cDNA being cloned with primers selected for IgG cloning as described in applicant's co-pending international application WO2008/081008, the disclosure content of which is incorporated herein by reference. Accordingly, respective antibodies or functionally active antibody fragments can be expressed and tested for selective binding properties.

Methods of producing clones of an immortalized human B cell and B memory lymphocyte, comprising the step of transforming human B memory lymphocytes using Epstein Barr Virus (EBV) in the presence of a polyclonal B cell activator are summarized in international application WO2004/076677, the disclosure content of which is incorporated herein by reference. This international application also describes methods for obtaining a nucleic acid sequence that encodes an antibody of interest, comprising the steps of preparing an immortalized B cell clone and obtaining/sequencing the nucleic acid from the B cell clone that encodes the antibody of interest and further inserting the nucleic acid into or using the nucleic acid to prepare an expression host that can express the antibody of interest, culturing or sub-culturing the expression host under conditions where the antibody of interest is expressed and, optionally, purifying the antibody of interest. It goes without saying that the nucleic acid may be manipulated in between to introduce restriction sites, to change codon usage, and/or to add or optimize transcription and/or translation regulatory sequences. All these techniques are state of the art and can be performed by the person skilled in the art without undue burden.

However, since initial attempts of cellular cloning of identified antigen-specific EBV-transformed human memory B cells had not been successful, the method of the present invention provides an important alternative to the prior art and performs even superior in its results compared to the method using EBV, since the resultant clones are more stable and seem to be less prone for somatic mutations affecting the immunoglobulin genes.

Thus, the method of the present invention greatly facilitates the cloning of human, in particular patient-derived antibodies. Applied to selected clinical responders it is expected that the method of the present invention will lead to the identification and isolation of novel candidate antibodies for the immunotherapy of various diseases as well as to the isolation of novel disease associated antigens which because of the selectivity and specificity of the method of the present invention may be more reliable for use as clinical markers and targets for therapeutic intervention.

In a preferred embodiment the method of the present invention comprises culturing the B lymphocyte in the presence of a polyclonal B cell activator. For example, the nucleic acid molecule encoding the polypeptide having telomerase activity is transfected in the presence of a polyclonal B cell activator. The term “polyclonal activator” means a molecule or compound or a combination thereof that activates B lymphocytes irrespective of their antigenic specificity. A range of different molecules may be used as the polyclonal activator and are known to the person skilled in the art; see, e.g., those described in international application WO2004/076677. The polyclonal B cell activator can be a CpG oligodeoxynucleotide among others. However, preferably CpG 2006 is used.

Additional stimulants of cellular growth and differentiation may be added during the transformation step to further enhance the efficiency. These stimulants may be cytokines such as IL-2 and IL-15. In a particularly preferred aspect, IL-2 is added during the induction step to further improve the efficiency of immortalization, but its use is not essential.

The B lymphocytes to be used in accordance with the present invention can come from various sources (e.g. from whole blood, from peripheral blood mononuclear cells (PBMCs), from blood culture, from bone marrow, from organs, etc.), and suitable methods for obtaining human memory B cells are well known in the art. Samples may include cells that are not memory B cells e.g. other blood cells. A specific human memory B lymphocyte subpopulation exhibiting the desired antigen specificity may be selected before inducing or enhancing telomerase activity by using methods known in the art.

In principle, any desired antigen may be selected including but not limited to the group consisting of a human pathogen, toxin, chemical compound, allergen, tumor antigen, autoantigen, alloantigen or neoepitope of an otherwise physiological protein. Antigens of interest are disclosed for example in international application WO2004/076677. Most preferably, the antigen is involved in Alzheimer's disease or cancer. In this context, the present specification is specifically supplemented with the teaching provided by international application WO2008/081008 and WO2008/110373 regarding the identification and isolation of beta amyloid (A3) peptide and tumor antigen specific human antibodies, respectively. In particular, the method for identifying, validating and producing Aβ peptide specific diagnostically and therapeutically useful binding molecules essentially as disclosed in international application WO2008/081008 may be employed but altered on the level of B cell immortalization as disclosed in the present application. Furthermore, regarding the isolation and molecular cloning and recombinant production of patient-derived human antibodies the present specification is supplemented by the method of screening of oligoclonal memory B cell cultures established from patient peripheral blood lymphocytes (PBLs) combined with a molecular cloning step using single cell RT-PCR and the re-screening of recombinant antibody clones with tissue microsections as disclosed in WO2008/110373. Likewise, the method for identifying, validating and producing tumor-specific diagnostically and therapeutically useful binding molecules, in particular human antibodies that are directed against antigens associated with tumor cells and tissue essentially as disclosed in international application WO2008/110373 may be employed but again altered on the level of B cell immortalization as disclosed in the present application.

Hence, in a particular preferred embodiment of the method of the present invention the B lymphocyte is derived from a sample obtained from a subject who is symptom-free but affected with or at risk of developing a disorder, or a patient with an unusually stable disease course. This embodiment is a further development of the corresponding methods of obtaining patient and disease specific human antibodies disclosed in international applications WO2008/081008 and WO2008/110373.

Accordingly, the present invention also relates to a method of isolating a disorder-associated protein-specific binding molecule, particularly a human antibody, comprising:
(a) subjecting a sample obtained from a patient who is symptom-free, or who is clinically unusually stable, but who is affected with or at risk of developing a disorder or effectively suppressing the manifestation or outbreak of a disorder to a specimen of pathologically or physiologically altered cells or tissue of predetermined clinical characteristics; and
(b) identifying and optionally isolating a binding molecule which preferentially binds to said specimen but not or with significantly lower affinity to corresponding cells or tissues without such pathological characteristics as it may be derived from a healthy subject;
   wherein the sample comprises B lymphocytes, preferably human memory B cells, which are treated so as to induce or enhance telomerase activity in the B lymphocyte as described herein. As for the steps of preamble of this embodiment of the present invention, these can be performed as outlined in the Examples sections in international applications WO2008/081008 and WO2008/110373 with means well known to a person skilled in the art

The present invention also provides an antibody and equivalent antigen-binding molecule obtainable by the method of the present invention, which antibody is preferably a human antibody having two polypeptide chains, wherein one or both of polypeptide chains has/have a human VDJ sequence. Monoclonal antibodies produced by the methods of the present invention may be further purified, if desired, using filtration, centrifugation and various chromatographic methods such as HPLC or affinity chromatography. Techniques for purification of monoclonal antibodies, including techniques for producing pharmaceutical-grade antibodies, are well known in the art.

Fragments of the monoclonal antibodies of the present invention can be obtained from the monoclonal antibodies so produced by methods that include digestion with enzymes, such as pepsin or papain, and/or by cleavage of disulfide bonds by chemical reduction. Antibody “fragments” include Fab, F(ab′)₂ and Fv fragments. The present invention also encompasses single-chain Fv fragments (scFv) derived from the heavy and light chains of a monoclonal antibody of the invention, e.g. the invention includes an scFv comprising the CDRs from an antibody of the invention.

For the sake of clarity only and without restricting the scope of the present invention most of the embodiments are discussed herein with respect to human antibodies and antibody-like molecules which represent the preferred binding molecules for the development of therapeutic and diagnostic agents in accordance with the present invention. However, it is to be understood that as used in context of the present invention the term “antibody”, and fragment thereof, may also refer to other non-antibody binding molecules including but not limited to hormones, receptors, ligands, major histocompatibility complex (MHC) molecules, chaperones such as heat shock proteins (HSPs) as well as cell-cell adhesion molecules such as members of the cadherin, integrin, C-type lectin and immunoglobulin (Ig) superfamilies. Monoclonal antibodies are particularly useful in identification and purification of the individual polypeptides or other antigens against which they are directed. The monoclonal antibodies of the invention have additional utility in that they may be employed as reagents in immunoassays, radioimmunoassays (RIA) or enzyme-linked immunosorbent assays (ELISA). In these applications, the antibodies can be labeled with an analytically-detectable reagent such as a radioisotope, a fluorescent molecule or an enzyme. The monoclonal antibodies produced by the above method may also be used for the molecular identification and characterization (epitope mapping) of antigens recognized by protected individuals in complex pathogens such as plasmodia, the isolation of cross-reactive protective antibodies in the case of highly variable pathogens such as those found in HIV and for detecting pathogens and determining their variability.

Antibodies of the present invention can be coupled to a drug for delivery to a treatment site or coupled to a detectable label to facilitate imaging of a site comprising cells of interest, such as cancer cells. Methods for coupling antibodies to drugs and detectable labels are well known in the art, as are methods for imaging using detectable labels. Antibodies of the invention may be attached to a solid support.

Antibodies of the invention are preferably provided in purified form. Typically, the antibody will be present in a composition that is substantially free of other polypeptides, e.g. where less than 90% (by weight), usually less than 60% and more usually less than 50% of the composition is made up of other polypeptides.

Antibodies of the invention may be immunogenic in non-human (or heterologous) hosts e.g. in mice. In particular, the antibodies may have an idiotope that is immunogenic in non-human hosts, but not in a human host. Antibodies of the invention for human use include those that cannot be obtained from hosts such as mice, goats, rabbits, rats, non-primate mammals, etc. and cannot be obtained by humanization or from xeno-mice.

Antibodies of the invention can be of any isotype (e.g. IgA, IgG, IgM, i.e. an α, γ or μ heavy chain), but will generally be IgG. Within the IgG isotype, antibodies may be IgG1, IgG2, IgG3 or IgG4 subclass. Antibodies of the invention may have a κ or λ, light chain.

The antibody of the present invention advantageously displays particularly high binding affinity with an equilibrium dissociation constant (KD) of the interaction with its cognate antigen in the lower nanomolar range. Preferably, the binding affinity of the binding molecule of the present invention with its cognate antigen is about at least 10⁻⁷M, more preferably at least 10⁻⁸M, particularly preferred 10⁻⁹M and still more preferred at least 10⁻¹⁰ M.

The present invention also provides a pharmaceutical and diagnostic, respectively, pack or kit comprising one or more containers filled with one or more of the above described ingredients, i.e. antibody or equivalent binding molecule derived thereof, or corresponding means for their production and/or delivery, for example a polynucleotide, particularly vector encoding the antibody or a cell containing the same. Associated with such container(s) can be a notice in the form prescribed by a governmental agency regulating the manufacture, use or sale of pharmaceuticals or biological products, which notice reflects approval by the agency of manufacture, use or sale for human administration. In addition or alternatively the kit comprises reagents and/or instructions for use in appropriate diagnostic assays.

The pharmaceutical compositions of the present invention can be formulated according to methods well known in the art; see for example Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intra-muscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

The above disclosure generally describes the present invention. Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2. Several documents are cited throughout the text of this specification. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

The practice of the present invention will employ, unless otherwise indicated, conventional techniques of cell biology, cell culture, molecular biology, transgenic biology, microbiology, recombinant DNA, and immunology, which are within the skill of the art. For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107.

### Supplementary Methods

### Memory B Cell Display

Memory B cells are isolated with a two step selection protocol using the pan B cell marker CD22 as a positive selection criteria, combined with negative selection of antigen-inexperienced B cells that expressed IgM, IgD. With this technique, approximately 10.000 to 100.000 memory B cells can be obtained from 30 ml of human blood.

These memory B cells are immortalized with hTert-expressing lentivectors and cultured oligo-clonally on irradiated human peripheral blood lymphocytes as feeder layers (Zubler et al., J. Immunol. 134 (1985), 3662-3668); Traggiai et al., Nat. Med. 10 (2004), 871-875. To improve transformation and immortalization efficacy of antibody-secreting memory B cells, CpG 2006 which mimics the activities of bacterial un-methylated CpG-dinucleotides as described by Hartmann and Krieg, J. Immunol. 164 (2000), 944-953, can be used.

### Experimental Protocol:

Selection of B cells from the bulk of PBL was performed using the MACS technology and CD22 microbeads (Miltenyi, Bergisch Gladbach, Germany). PBL were labeled with MACS anti human CD22, phycoerythrin-conjugated mAbs anti human IgD and APC-conjugated antibodies anti human IgM, CD3, CD8, CD56 (Becton Dickinson, Basel, Switzerland). CD22-positive cells were isolated using LS columns and the Midi MACS device (Miltenyi) followed by selection of phycoerythrin- and APC-negative cells using a MoFlo cell sorter (Dako, Fort Collins, USA). CD22-positive, IgM-, IgD-negative B cells were then incubated with hTert-expressing lentivector containing conditioned medium obtained from transfected 293T HEK cells and CpG 2006 (Sigma, Buchs, Switzerland) at a concentration of 2.5 mg/l in B cell medium (RPMI 1640 supplemented with 10% fetal calf serum (Hyclone, Perbio, Lausanne, Switzerland). 20 cells were seeded per well in Costar round bottom 96 well plates (Corning, Vitaris, Baar, Switzerland) in B cell medium on 30.000 irradiated human PBL prepared from voluntary donors. Memory B cell cultures were maintained at 37° C. and 5% Co₂ in a humidified cell culture incubator for 2-4 weeks after which time the conditioned medium of the cultures was assayed in ELISA and tissue arrays.

### Lentivectors

The catalytic subunit of human telomerase, was re-cloned from a hTert-expressing onco-retroviral vector described by Rufer et al. Blood 98 (2001), 597-603, into a 3^{rd} generation lentivector transfer vector described by Dull et al. Journal of Virology 72(11) (1998), 8463-71. In this CMV-hTert/SV-40-EGFPpRRL vector the expression of hTert was driven by the CMV promoter. GFP-expression which was necessary for lentivector titer determination, was driven by the SV-40 promoter. Infective lentivector particles were generated by co-transfection of transfer vector CMV-hTert/SV-40-EGFPpRRL, the core packaging plasmid pMDLg/pRRE, the envelope plasmid pMD2-VSV-G and the REV expressing plasmid pREV into 239T cells as described by Dull et al. (1998), supra. Conditioned medium of transfected 293 cells containing lentivectors was collected on day 2 after transfection. Lentivector titers were determined upon transduction of 293 HEK cells using serial dilutions of virus by measuring the proportion of 293 HEK cells that expressed lentivector encoded green fluorescent protein.

Transfection of Memory B Cells (mBC)

mBC were incubated with lentivector at a virus concentration of 50.000 efu/μl for 1 h in 200 ul of B cell medium supplemented with 2.5 mg/l CpG 2006 (Sigma, Buchs, Switzerland) in 5 ml round bottom tubes (Falcon BD, Allschwil CH). After this incubation the volume was increased by addition of 200 ul of B cell medium containing 2.5 μg/ml CpG 2006 and incubation was continued for 3 more hours. Cells were seeded in 96 well microtiter templates at 20 mBC/well on irradiated feeder cells (30.000/well) in RPMI 1640 supplemented with 10% fetal calf serum and 2.5 μg/ml CpG 2006.

### ELISA

96 well half area Microplates (Corning) were coated with TT at a standard concentration 0.4 μg/ml in coating buffer (15 mM Na₂CO₃, 35 mM NaHCO₃, pH 9.42) overnight at 4° C. Plates were washed and non-specific binding sites were blocked for 1 h at RT with PBS containing 2% BSA (Sigma, Buchs, Switzerland). B cell conditioned medium was transferred from memory B cell culture plates to ELISA plates and was incubated for 2 h at room temperature. Binding of human antibodies was determined using horse radish peroxidase (HRP)-conjugated donkey anti-human IgG polyclonal antibodies (Jackson ImmunoResearch Europe Ltd., Cambridgeshire, UK) followed by measurement of HRP activity in a standard colorimetric assay.

### Cellular Cloning

Cloning was performed by single cell deposition into 96 well culture plates using a cell sorter (MoFlo, Dako, Fort Collins, USA) the device was set to deposit one single cell (single 1 mode) per well directly in 96 well plates filled with B cell medium and 30.000 irradiated feeder cells.

### PCR-ELISA Telomeric Repeat Amplification Protocol (TRAP)

Telomerase activity in human B cell lines was measured using the TeloTAGGG PCR-ELISA (Roche Diagnostics, Rotkreuz, Switzerland) a photometric enzyme immunoassay based on the telomeric repeat amplification protocol (TRAP). The assay was performed according to the manufacturer's instructions. The photometric reaction was analyzed using a standard ELISA reader (TECAN Sunrise, Tecan, Switzerland)) at 450 nm and using 690 nm as reference wavelength.

### Example 1

### Immortalization and Preservation of a Clonally Diverse B Cell Population Human Memory B Cells

As a strategy to immortalize B cells, the ectopic expression of hTert was chosen. As a transducing vector for hTert expression in human B cells lentiviral vectors derived from HIV1 were used. Lentiviral vectors used in the experiments were members of the so-called 3^{rd} generation of lentivectors, originally designed for in vivo gene therapy. They are offering the most advanced safety features available to date (Zufferey et al., J. Virol. 72 (1998), 9873-9880). The lentivectors were pseudotyped with the vesicular stomatitis virus glycoprotein. Vectors of this pseudotype have been shown previously to be able to transduce human B cells depending on their simultaneous activation with an appropriate stimulus (Bovia et al., Blood 101 (2003), 1727-1733). The transduction efficiency could be further improved by a stimulation of human B cells with CpG-oligodeoxynucleotides (Kvell, et al., Mol Ther 12(5) (2005), 892-899).

The capability of hTert-expressing lentivectors to immortalize human memory B cells was evaluated by transducing peripheral blood memory B cells that were selected based on their expression of the pan-B cell surface marker CD22 and the absence of surface immunoglobulin M and D. CD22^{+/IgM}⁻/IgD⁻-cells were co-incubated with concentrated hTert-lentivectors at a concentration of 50.000 expression forming units (efu/ul) in a small volume for 4 h in complete medium supplemented with CpG 2006. After a lag phase of 4 weeks the cells began to proliferate and were passaged for over 2 months after which period the cells were cryopreserved.

A thawed aliquot of the cells was propagated further and the clonality of cells was determined upon molecular cloning of the Ig-variable heavy chain region and subsequent sequence analysis. To that end mRNA obtained from an aliquot of cells was reverse transcribed and the Ig-heavy variable regions were amplified by PCR using frame work 1 and J-H-region specific primer mixes specific for all families of the human Ig-variable heavy chain. After cloning into appropriate plasmid vectors and transformation into E. coli the sequences of 10 randomly picked clones were analyzed. Six clones showed identical Ig-Vh-sequences, whereas four other clones were unique. Thus, despite the presence of a dominant clone, clonal diversity was maintained throughout the transformation and proliferation process. This suggests that the proliferating cells that were obtained after co-incubation of memory B cells (mBC) with hTert were not the result of the expansion of singular, rare immortalization event but rather that transformation occurred on a broad clonal basis.

### Example 2

### Secretion of Human IgG of hTert-Lentivector-Transduced B Cells

As a next step in the evaluation of hTert-lentivector transduced mBC their capability to secrete IgG was assayed. Memory B cells do not secrete IgG under physiological conditions and, in culture, only upon transformation induced by stimulation with the appropriate cytokines, cell-cell contact or upon transduction and transformation by EBV as described by Steinitz et al., Nature 287 (1980), 443-445; Zubler et al., J. Immunol. 134 (1985), 3662-3668; Tew et al., Immunol. Rev. 126 (1992), 99-112; Bernasconi et al., Science 298 (2002), 2199-2202.

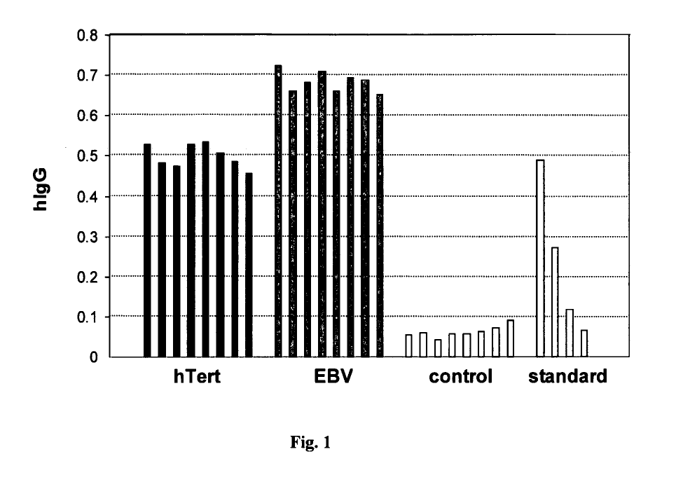

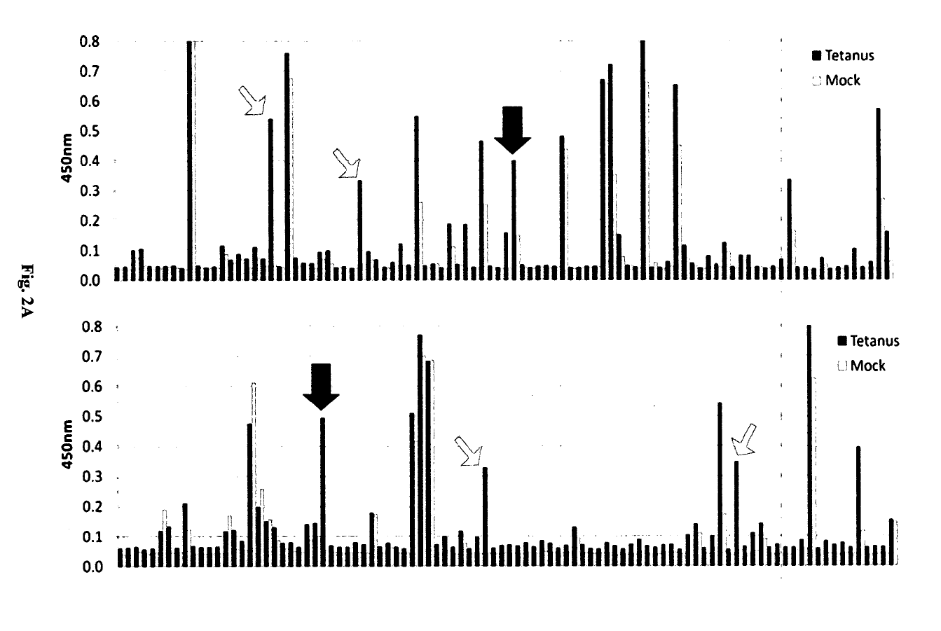

In order to assay the secretion of immunoglobulins, human memory B cells were co-incubated with hTert-expressing lentivector and 20 memory B cells were seeded per well in 96 well plates on a layer of irradiated feeder cells. In this assay, proliferation became manifest after 3-4 weeks of culture. IgG production of was assayed after this culture period in comparison with that secreted by EBV transduced mBC or cultures of non-transduced mBC that had also been incubated on irradiated feeder cells. As shown in FIG. 1 cultures of memory B cells transformed by hTert-expressing lentivector secrete IgG at levels that are comparable with that of EBV immortalized mBC.

### Example 3

### Secretion of TT-Specific Antibodies in mBC and Cloning of TT-Specific hTert-Lentivector-Transduced B Cells

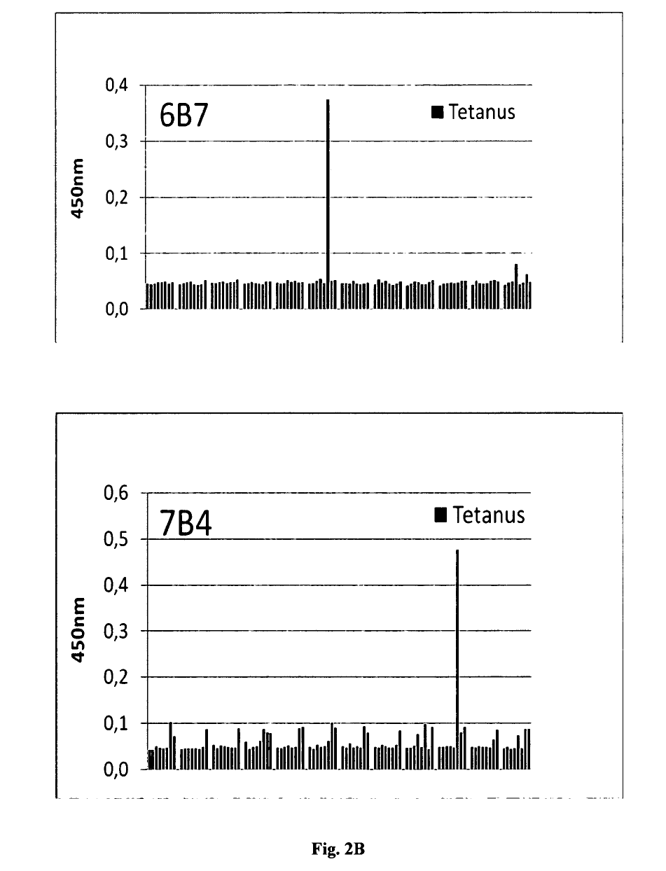

Tetanus toxoid (TT) was selected as surrogate antigen of interest and memory B cell cultures secreting an IgG specific to TT were detected by assaying the medium conditioned by the cultures growing in a 96 well plate in ELISA using TT coated plates (FIG. 2A). Non-specific binding was assessed in a second ELISA using mock coated (BSA) plates.

Two cultures containing antibodies specific for TT, 6B7 and 7B4, were selected for further propagation in 24 well plates for 1 week. Clonal lines were obtained from these cultures upon cloning using single cell deposition into 96 well plates with a cell sorter and sub-sequent re-screening of the medium conditioned by clonally growing cells for reactivity in TT-ELISA. With both cultures 6B7 and 7B4, one clonal cell line still expressing TT-specific antibodies was obtained after a culture period of four weeks demonstrating the possibility that the transformation of memory B cells by hTert-lentivector significantly extended the life span of human memory B cells (FIG. 2B) and supported their clonability.

### Example 4

### Human Telomerase Activity in Memory B Cells Treated with hTert-Lentivector

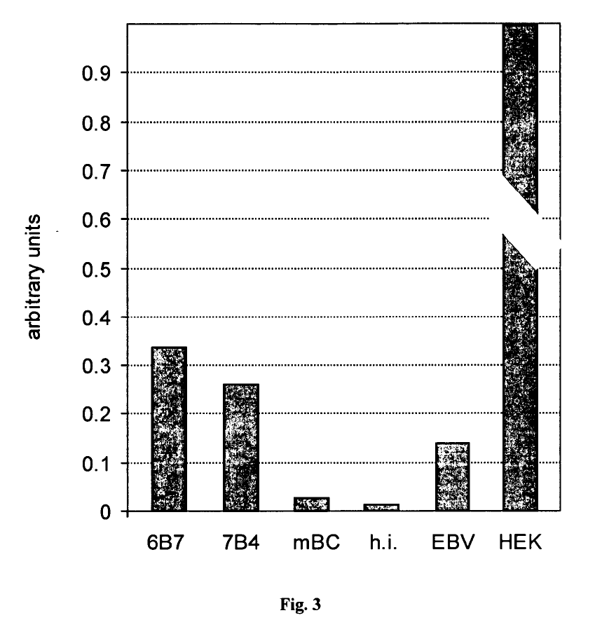

The telomerase activity of clonal B cell lines transduced with hTert-lentivector was assayed in comparison with that of EBV-transformed memory B cells using a semiquantitative telomerase PCR ELISA assay. Cellular extracts from 200.000 memory B cells (100.000 for clonal line 6B7) were assayed for their ability to prolong an artificial telomerase substrate DNA. In the two hTert-lentivector transduced B cell lines tested a significantly higher Telomerase activity was observed as compared to that of freshly isolated memory B cells or a EBV-transformed B cell line (FIG. 3). This supports the notion, of a transgene-mediated over-expression of hTert being at the origin of the extension of life span observed with hTert-lentivector transduced human memory B cells.

1. A method of preparing a monoclonal antibody or equivalent antigen-binding molecule comprising producing a B lymphocyte of prolonged life span by inducing or enhancing telomerase activity in the B lymphocyte.

2. The method of claim 2, wherein the B lymphocyte is a human memory B cell.

3. The method of claim 1, wherein
the induced or enhanced telomerase activity is due to the presence of a foreign nucleic acid molecule or polypeptide in the B lymphocyte.

4. The method of claim 1, comprising introducing into the B lymphocyte a nucleic acid molecule encoding a polypeptide having telomerase activity.

5. The method of claim 4, wherein the polypeptide having telomerase activity is Telomerase-Reverse-Transcriptase (Tert), or a catalytically active fragment or derivative thereof.

6. The method of claim 3, wherein the nucleic acid molecule is contained in a vector.

7. The method of claim 6, wherein the vector is a lentiviral vector.

8. The method of claim 1, comprising culturing the B lymphocyte in the presence of a polyclonal B cell activator.

9. The method of claim 4, wherein the nucleic acid molecule is transfected in combination with a polyclonal B cell activator.

10. The method of claim 8, wherein the polyclonal B cell activator is a CpG oligodeoxynucleotide, preferably CpG 2006.

11. The method of claim 1, further comprising culturing the B lymphocyte in the presence of a stimulant of cellular growth and/or differentiation.

12. The method of any one of claim 11, wherein the stimulant is a cytokine, preferably IL-2 or IL-15.

13. The method of claim 5, wherein a subpopulation of B lymphocytes having antigen specificity is selected before inducing or enhancing telomerase activity.

14. The method of any one of claim 13, wherein the antigen is selected from the group selected of a human pathogen, toxin, chemical compound, allergen, tumor antigen, autoantigen, alloantigen or neoepitope of an otherwise physiological protein.

15. The method of claim 1, wherein the B lymphocyte is derived from a sample obtained from a subject who is symptom-free but affected with or at risk of developing a disorder, or a patient with an unusually stable disease course.

16. The method of claim 1, comprising subsequent identification and cloning of B lymphocytes that produce an antibody.

17. The method of claim 1, comprising producing a clone of a substantially immortalized human memory B cell capable of producing a human monoclonal antibody with a desired antigen specificity, comprising the steps of:
(i) transforming a population of cells comprising or consisting of human memory B lymphocytes with a Lentivirus virus (EBY) encoding a polypeptide providing
Telomerase-Reverse-Transcriptase activity in the presence of a polyclonal B cell activator;
(ii) screening the culture supernatant for antigen specificity; and
(iii) isolating a human memory B cell clone of prolonged life time for several or multiple cycles of replication capable of producing a human monoclonal antibody having the desired antigen specificity.

18. The method of the claim 16, wherein the cloning is carried out using limiting dilution.

19. A substantially immortalized B lymphocyte clone obtainable by the method of claim 1.

20. The method of claim 1 further comprising the steps of: (i) purifying the B lymphocytes from a sample which has been identified to express an antibody of desired specificity; (ii) obtaining the immunoglobulin gene repertoire for said antibody from the B lymphocytes; and
(iii) using said repertoire to express the antibody.

21. The method of claim 20, wherein step (ii) comprises the steps of (iv) obtaining mRNA from the B lymphocytes;
(v) obtaining cDNA from the mRNA of step (iv); and
(vi) using a primer extension reaction to amplify from said cDNA the DNA fragments corresponding to the immunoglobulin heavy chains (HC) and the kappa light chains (LC) of the antibody.

22. The method of claim 21, further comprising inserting the DNA fragment into an expression host cell in order to permit expression of the antibody of interest or immunoglobulin chain thereof in that host cell.

23. The method of claim 22, wherein the nucleic acid molecule is manipulated between steps (ii) and (iii) to introduce restriction sites, to change codon usage, alter the amino acid sequence of the immunoglobulin chain while keeping antigen specificity in kind and/or to add or optimize transcription and/or translation regulatory sequences.

24. The method of claim 22, further comprising culturing the host cell under conditions where the antibody of interest is expressed; and optionally purifying the antibody the interest or immunoglobulin chain thereof.

25. The method of claim 1, comprising the steps of:
(a) transforming a human memory B cell into a substantially immortalized immunoglobulin secreting cell, comprising (i) introducing human Telomerase-Reverse-Transcriptase (hTert) through lentivector-mediated gene transfer into human blood-derived memory B cells;
(ii) in the presence of a polyclonal B cell activator; (b) selecting a B cell of prolonged life span that produces an antibody with a desired specificity;
(c) obtaining and/or sequencing a nucleic acid molecule from the selected B cell encoding at least the binding domain of the antibody of interest;
(d) inserting the nucleic acid molecule into or using the nucleic acid sequence to prepare an expression host cell that is capable of expressing the antibody of interest or an immunoglobulin chain thereof;
(e) culturing or sub-culturing the expression host cell under conditions where the antibody of interest is expressed; and, optionally,
(f) purifying the antibody of the interest or immunoglobulin chain thereof.

26. An antibody or equivalent antigen-binding molecule obtainable by the method of claim 1.

27. The antibody of claim 26, which is a human antibody.

28. (canceled)

29. (canceled)

30. (canceled)
